(19) United States
(12) Patent Application Publication
(10) Pub. No.: US 2006/0057192 A1
(43) Pub. Date: Mar. 16, 2006

(54) Localized non-invasive biological modulation system

(76) Inventor: Patrick D Kane, Cambridge, MA (US)

Correspondence Address: Ronald I Eisenstein;Nixon Peabody, 100 Summer Street, Boston, MA, 02110, US

(21) Appl. No.: 10/510,518

(22) PCT Filed: Sep. 30, 2002
(86) PCT No.: PCT/US02/30997

§ 371 Date: Aug. 1, 2005

Related U.S. Application Data
(60) Provisional application No. 60/325,673, filed on Sep. 28, 2001.
(60) Provisional application No. 60/364,734, filed on Mar. 14, 2002.

(51) Int. Cl. A61K 9/127

(52) U.S. Cl. 424/450

(57) ABSTRACT

The present invention provides methods for non-invasive localized delivery of biologically active molecules, comprising packaging a molecule(s) of interest inside a thermosensitive particle, administering said particles to a subject, and inducing localized release of said molecules from said particles using a focused heat source. The thermosensitive particles may be thermosensitive polymer nanoparticles or thermosensitive liposomes. The particles may be delivered to a subject by any technique, including infusion. The molecules may be released from the particles using any method which induces localized hyperthermia, including focused ultrasound.

### FIELD OF THE INVENTION

The present application is directed to methods for localized non-invasive delivery of biological modulating agents throughout the body, particularly for the delivery of neuromodulators to specific sites within the brain.

### BACKGROUND OF THE INVENTION

There are a range of methods of delivery of an agent to a subject. For in vivo administration, methods include catheters, injection, scarification, etc. For example, stereotaxic injection can be used to direct delivery of an agent to a desired location in the brain. Stereotaxic surgery is performed using standard neurosurgical procedures [Pellegrino and Clapp, Physiol. Behav. 7: 863-8 (1971)]. Additionally, agents can be delivered by intracerebroventricular (“icv”) infusion using a minipump infusion system, such as a SynchroMed Infusion System. A recent method based on bulk flow, termed convection, has also proven effective at delivering large molecules to extended areas of the brain and may be useful in delivering the viral particle to the target cell [R. Bobo et al., Proc. Natl. Acad. Sci. USA 91: 2076-80 (1994); P. Morrison et al., Am. J. Physiol. 266: R292-305 (1994)]. Other methods can be used including catheters, intravenous, parenteral, intraperitoneal and subcutaneous injection, oral, inhalation, or other known routes of administration.

However, many of these methods are systemic, or at best regional in application. This can result in delivery of an agent to normal tissues, where the effect of the agent can be deleterious. Thus, a method for targeted delivery of an agent to only a particular region would be desirable. It would also be desirable to do this in as non-invasive a manner as possible. Accordingly, localized targeted drug delivery is highly desirable for a wide array of applications. For example, the function of the central nervous system relies on the interconnectivity of specific subsets of neurons, which communicate using many different neurotransmitters. Many neurodegenerative diseases are characterized by loss of function of these connections, known as synapses. For example, Parkinson's Disease is a loss of dopaminergic activity in the pigmented neurons of the substantia nigra. Thus, it would be highly desirable to be able to deliver agents including drugs, genes, etc. in a non-invasive manner to a very specific site.

The brain presents particular needs and challenges for targeted drug delivery. For example, the ability to excite or inhibit the activity of specific subsets of neurons in specific regions of the brain. The inability of many agents to cross the blood-brain barrier also causes problems.

While sophisticated techniques for drug delivery have been developed, there remains a need for improved methods for the precise localized deposition of biologically active agents. Many existing methods rely on invasive techniques, such as localized injection to deliver an agent to its site of action. Even then the agent may disperse from that site. Moreover, such techniques are inherently fraught with the risks of infection associated with any invasive procedure. Furthermore, certain tissues, such as the brain, are particularly sensitive to any intervention. Thus, it would be highly desirable to have a non-invasive method for the localized delivery of agents.

One advance in drug delivery has been the development of liposomes, including time-release liposomes and thermosensitive liposomes. Liposomes consist of at least one lipid bilayer membrane enclosing an aqueous internal compartment. Conventional liposomes are formulated to carry therapeutic agents, drugs or other active agents either contained within the aqueous interior space (water soluble active agents) or partitioned into the lipid bilayer (water-insoluble active agents). Active agents that have short half-lives in the bloodstream are particularly suited to delivery via liposomes. Many anti-neoplastic agents, for example, are known to have a short half-life in the bloodstream such that their parenteral use is not feasible. However, the use of liposomes for site-specific delivery of active agents via the bloodstream is limited by the rapid clearance of liposomes from the blood by cells of reticuloendothelial system (RES).

Liposomes are normally not leaky but will become so if a hole occurs in the liposome membrane, if the membrane degrades or dissolves, or if the membrane temperature is increased to the phase transition temperature. The elevation of temperature (hyperthermia) at a target site in a subject to raise liposome temperature above the phase transition temperature, and thereby cause the release of the liposome contents, has been used for the selective delivery of therapeutic agents. Yatvin et al., Science 204:188 (1979). Recently liposome formulations capable of delivering therapeutic amounts of active agents in response to mild hyperthermic conditions have been described (U.S. Pat. No. 6,200,598).

Thermosensitive liposomes have been developed which retain their structure at 37° C., human body temperature, but are destroyed at even slightly elevated temperatures (e.g. 42° C.). Microwaves have been used for localized drug delivery by spatial localized destruction of thermosensitive liposomes (for example to treat tumors in the hand). However, microwaves do not offer a high degree of localization. Thus, in situations where precise control is desired, for example when targeting specific regions of the brain, it is not satisfactory. Thermosensitive liposomes have also been used with an invasive source of heat for localized drug delivery. However, as described above, such invasive techniques are associated with infection risks and are not available for all regions of the body.

The administration of antineoplastic or antitumor drugs such as doxorubicin, cisplatin and methotrexate using thermosensitive liposomes in combination with hyperthermia at the desired target site has been reported. See, e.g., Magin and Weinstein In: Liposome Technology, Vol. 3, (Gregoriadis, G., ed.) p. 137, CRC Press, Boca Raton, Fla. (1993); Gaber et al., Intl. J. Radiation Oncology, Biol. Physics, 36(5):1177 (1996).

One approach which has been taken for localized delivery of therapeutic compounds is the use of gaseous precursor-filled microspheres, as described for example in U.S. Pat. No. 6,443,898. In this system, the gas in the microspheres expands when the microspheres are heated to a certain temperature, rupturing the microsphere and releasing the compounds contained within. Ultrasound, microwaves, magnetic induction oscillating energy, and light energy can be used to raise temperatures in a localized manner to rupture the microspheres. However, this system is associated with several important disadvantages, including the size of the microspheres, which typically have a diameter in the range of microns rather than nanometers. Such a large size restricts the utility of this method. In addition, the walls of the microspheres are typically comprised of lipids and/or polymers. Particularly considering their size, the microspheres are not readily available to modifications which allow them to be transported across the blood brain barrier.

Focused ultrasound has been recently reported for breaching the blood brain barrier (BBB), as described in Hynynen et al., Radiology 220:640-6 (2001). In this system, focused ultrasound was used to rupture microbubbles deep within the brain, causing a physical disruption of the BBB, thus allowing any material in the region of the rupture to non-selectively cross the BBB. While this work demonstrates the ability of focused ultrasound to access deep brain regions, it does not allow selective transport of a desired agent across the BBB.

Photolytic uncaging and microwaves have also been used for drug delivery. The science of photolyctic uncaging is another method for releasing biologically active agents in spatially and temporally restricted tissue. This method relies on electromagnetic energy as its focused deposition method. Unfortunately, the only wavelengths applicable to this process not strongly absorbed by some endogenous molecules are near-infrared and microwaves. Ultraviolet is most often used for photolytic uncaging; however, it is incapable of penetrating more than a millimeter or less into biological tissue thus is restricted to in vitro model use. Near-infrared can penetrate into tissue upwards of 20 centimeters, but is impossible to focus due to a severe scattering affect. Microwaves are another alternative to ultrasound for transcranial and deep brain energy deposition; however penetrating wavelengths in this domain can not be focused as well as ultrasound, i.e. reduced resolution. In addition, another important barrier for use of microwaves is their association with the potential carcinogenic effects; it has been extensively documented that prolonged exposure to microwaves may cause cancer.

Transcranial Magnetic Stimulation (TMS) represents another potential route for drug delivery. TMS has already been used for a number of applications, including increased learning ability in mammalian cortex (enhanced Long-Term Potentiation in networks activated while under the influence of the excitatory effects of localizaed TMS) and alleviation of depression, which have been demonstrated in vivo. TMS stimulates targeted brain regions by way of a noninvasive magnetic field. However, TMS is unable to penetrate beyond superficial brain layers, and it is only applicable to limited electrical excitation; it cannot be used to suppress activity, nor can it be used for drug delivery.

Brachytherapy, also known as targeted internal radiation, is an extremely new technology recently approved by the FDA to treat breast cancer. A spaghetti-like hollow catheter with an inflatable balloon is implanted at the tumor site after the tumor is removed by a traditional lumpectomy surgical procedure. Later, a radioactive seed is inserted through the catheter, and a targeted dose of radiation emits through the balloon. Although this treatment demonstrates the power of being able to deliver an anti-cancer agent such as radiation in a highly localized manner, it relies on an invasive surgical technique and an implanted device, which are associated with the risks of infection outlined above.

Micro-Electro-Mechanical Systems (MEMS) is the integration of mechanical elements, sensors, actuators, and electronics on a common silicon substrate through the utilization of microfabrication technology. The recent application of this technology to medical therapeutics is based on its ability to control the release of drugs in the localized region in which it is surgically implanted. However, this is again an invasive technology, demonstrating the need for a non-invasive method to deliver biologically active agents in a localized manner.

Targeting specific tissues using antibody conjugates is another approach taken to localize delivery of therapeutic agents. Unfortunately, tissue targeting does not necessarily mean spatially restricted anatomical localization. In the case of cancer, for example, targeting one form of cell would mean the destruction of not only the cancerous tumor, but also healthy tissue of the same form throughout the body. Furthermore, a novel antibody would be needed for each disorder, drastically increasing the difficulty of overall success and immensely reducing its therapeutic value and platform applicability.

Thus, there remains a need for improved methods of localized drug delivery. In particular, it would be highly desirable to have a non-invasive method for the localized delivery of biologically active molecules.

### SUMMARY OF THE INVENTION

The present invention provides methods for non-invasive localized delivery of biologically active molecules, comprising packaging a molecule(s) of interest inside an ultrasound-responsive particle, such as a thermosensitive particle, administering said particles to a subject, and inducing localized release of said molecules from said particles using a focused energy source.

Particles of the invention include thermosensitive nanoparticles and thermosensitive liposomes. Preferred thermosensitive nanoparticles include thermosensitive nanovesicles and thermosensitive nanospheres.

The present invention provides for administration of the particles to a subject by any technique, including oral and intravenous administration.

The molecules may be released from the particles using any non-invasive method which induces localized hyperthermia, including focused ultrasound.

Another preferred embodiment of the invention provides a nanosphere encapsulated within a thermosensitive particle, including within a thermosensitive polymersome or nanovesicle. In this embodiment, the nanosphere preferably contains a substance, such as a biologically active substance, for release over an extended period of time. Preferably, the substance is released for days; even more preferably, for weeks; even more preferably, months.

One preferred embodiment of the present invention provides methods for treating neural conditions. In this embodiment, the particles preferably are coated with any composition which promotes or enhances transport of the particles across the blood brain barrier, including overcoating the particles with Polysorbate 80/85 or coating the particles with antibodies which allow transport across the blood brain barrier. One preferred antibody is an anti-transferrin receptor antibody.

Neuromodulators include molecules which activate or inhibit specific populations of neurons. Preferred neural conditions include epilepsy, Alzheimer's disease, Parkinson's disease, stroke, developmental learning disabilities, and post-traumatic neuronal cell loss.

Other preferred embodiments of the present invention provide methods for treating arthritis.

Another embodiment of the present invention provides a method for targeted adipose tissue destruction.

One preferred embodiment of the present invention provides a method for targeted gene therapy.

### BRIEF DESCRIPTION OF THE DRAWINGS

FIG. 1 is a detailed depiction of a liposome.

FIG. 2 depicts non-invasive neuronal modulation.

FIG. 3A depicts another alternative nanoparticle configuration, in which the desired agent is embedded directly in a polymer engineered to cause content release at a desired temperature, as shown in FIG. 3B.

FIG. 4 depicts the nanosphere of FIG. 3, to which a conjugate or coating for targeting purposes has been added.

FIG. 5A depicts the basic configuration of a nanovesicle, consisting of a aqueous core containing the desired agent (such as a neurotransmitter or drug) encapsulated by a membrane-block copolymer engineered to cause controlled, localized content release at a desired temperature, as depicted in FIG. 5B, with the entire nanovesicle coated with polysorbate 80/85 to enhance transport efficacy across the blood brain barrier (BBB).

FIG. 6 depicts an alternative configuration for the nanovesicle depicted in FIG. 5, to which a targeting conjugate or coating has been added.

FIG. 7 depicts another alternative nanovesicle configuration, in which subvesicles or subparticles are encapsulated in a nanovesicle.

FIG. 8A depicts a fusible liposome subvesicle encapsulated within the aqueous core of a nanovesicle. FIG. 8B depicts the delivery of the liposome's contents to a cell via membrane fusion.

FIG. 9 depicts another alternative nanovesicle configuration in which a desired agent is encapsulated in a polymer engineered for timed release and/or endocytic delivery within a nanovesicle engineered to cause content release at a desired temperature.

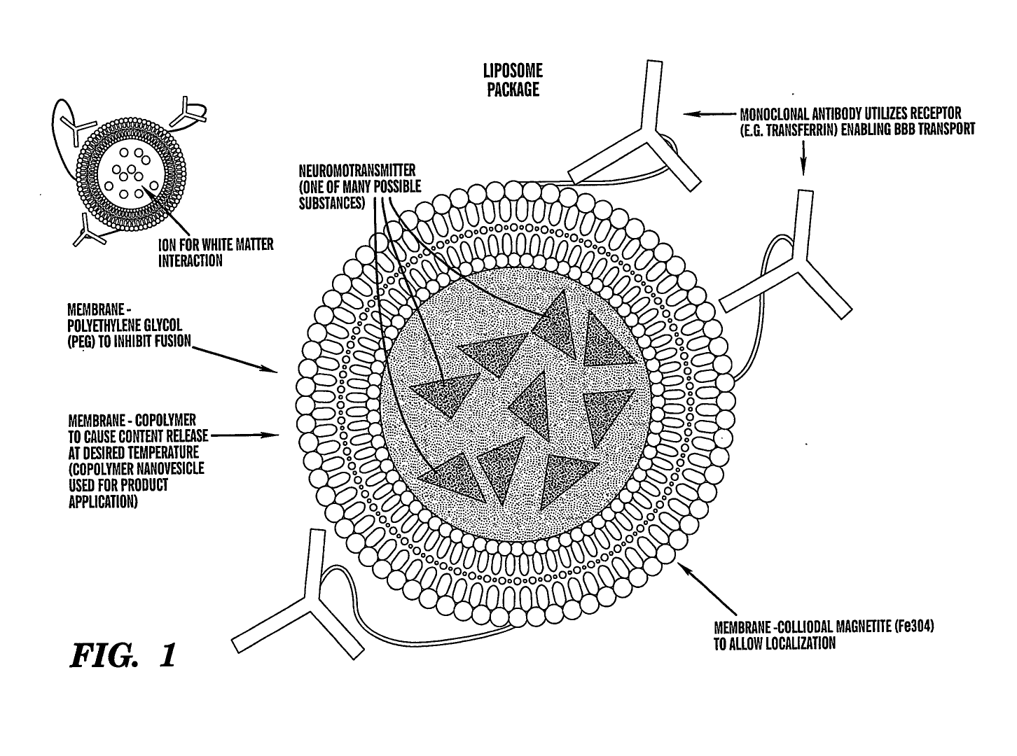

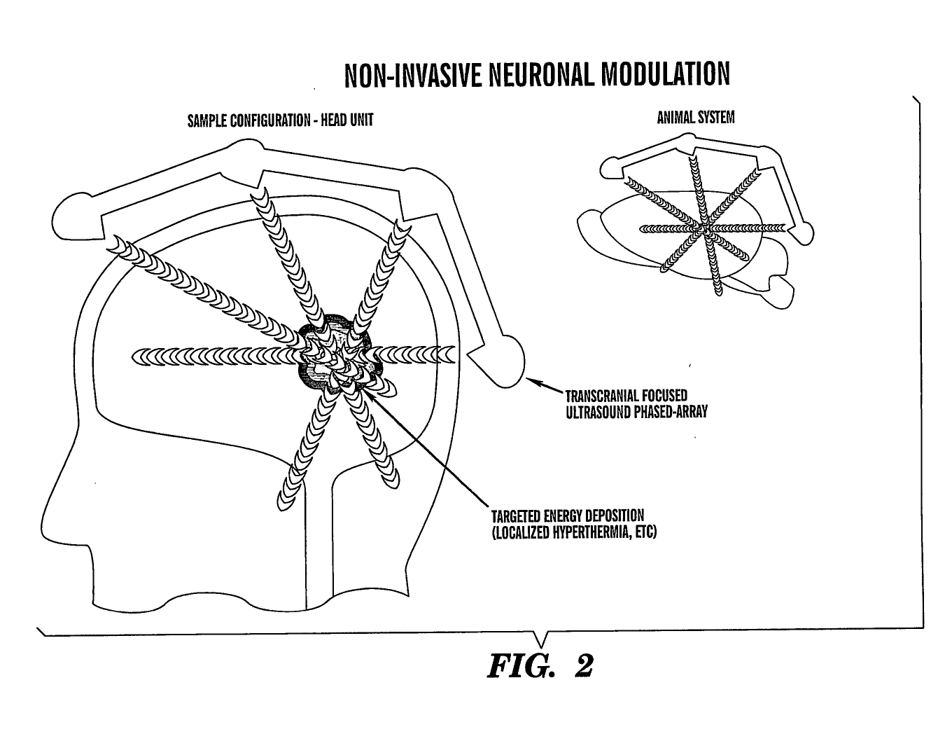

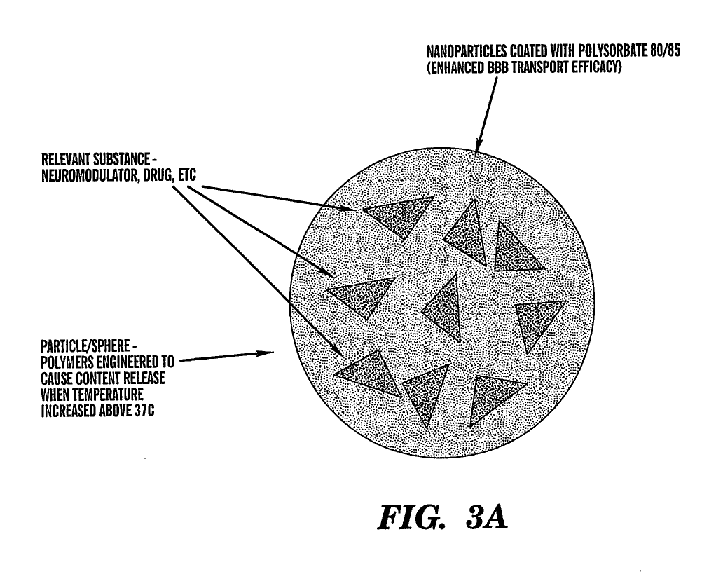

### DETAILED DESCRIPTION OF THE INVENTION

We have now discovered methods for non-invasive localized delivery of biologically active molecules, comprising packaging a molecule(s) of interest inside an ultrasound-responsive particle, such as a thermosensitive particle, administering said particles to a subject, and inducing localized release of said molecules from said particles using a focused energy source.

Thermosensitive Particles

Any thermosensitive particle which can package a molecule of interest and which is intact at body temperature (i.e. 37° C.) but destroyed at any other, non-body temperature which can be tolerated by a subject may be used. Particles of the invention include but are not limited to thermosensitive nanoparticles, thermosensitive polymersomes, and thermosensitive liposomes. Preferred thermosensitive nanoparticles include thermosensitive nanovesicles and thermosensitive nanospheres.

As used herein, nanoparticles include but are not limited to nanovesicles and nanospheres. Nanoparticles of the invention are sometimes referred to as thermosensitive nanoparticles or simply nanoparticles. One preferred nanoparticle is a thermosensitive polymer nanoparticle, which is sometimes referred to as a polymer nanoparticle. Another preferred nanoparticle is a lipid-polymer hybrid.

Thermosensitive nanovesicles include but are not limited to vesicles which are no smaller than 150 nanometers. Nanovesicles typically include a cavity which may contain any substance of interest. Substances which may be encapsulated within nanovesicles include but are not limited to biologically active agents, gases, and nanoparticles. One preferred nanovesicle of the invention is a nanoscale polymersome. Another preferred embodiment of the invention provides a polymer-lipid hybrid nanovesicle. Nanovesicles of the invention are sometimes referred to as thermosensitive polymer nanovesicles or polymer nanovesicles or thermosensitive nanovesicles or simply nanovesicles.

Thermosensitive nanospheres include but are not limited to spheres which are no smaller than 5 nanometers. Nanospheres typically do not contain a cavity. A substance of interest, such as a biologically active agent, may be incorporated into a nanosphere. Nanospheres of the invention are sometimes referred to as thermosensitive nanospheres or simply nanospheres.

Thermosensitive polymersomes include but are not limited to any polymer vesicle, including microvesicles and nanovesicles. As used herein, nanovesicles include nanoscale polymersomes.

Thermosensitive liposomes include but are not limited to any liposome, including time-release liposomes.

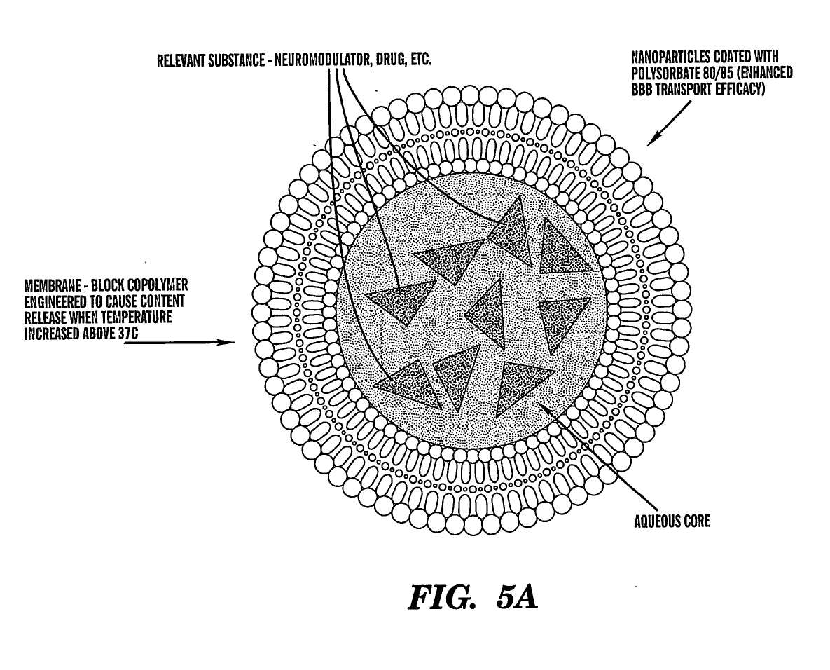

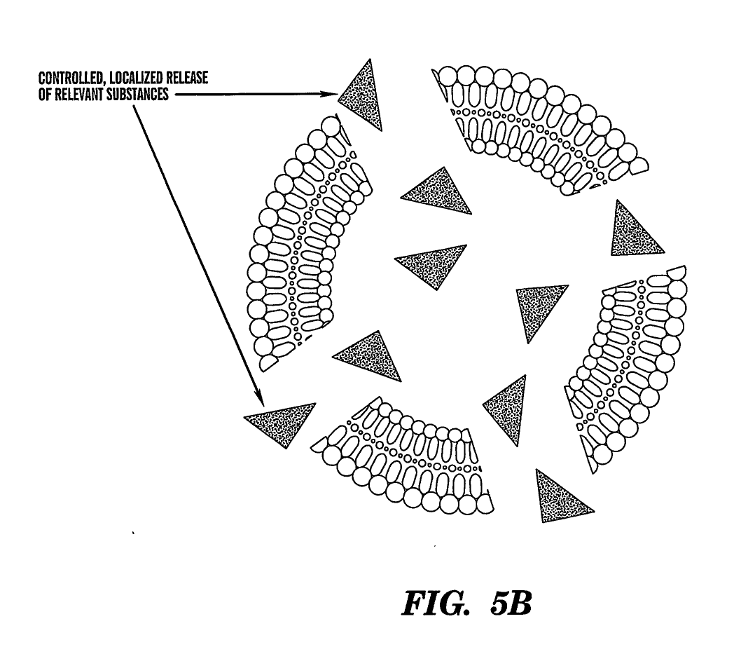

A preferred particle of the invention is a polymeric nanoparticle, including nanospheres, nanovesicles, and polymersomes as depicted in FIG. 5, consisting of a aqueous core containing the desired agent (such as a neurotransmitter or drug) encapsulated by a membrane-block copolymer engineered to cause content release at a desired temperature.

One preferred embodiment of the invention provides methods for treating neural conditions. In this embodiment, the particle is preferably coated with a composition to promote or enhance transport of the particles across the blood brain barrier (BBB). Preferably, the particle is overcoated with polysorbate 80/85 to enhance transport efficacy across the blood brain barrier. In another preferred embodiment, the particle is coated with an antibody which promotes or enhances transport across the blood brain barrier. Such antibodies are known in the art and include but are not limited to an anti-transferrin receptor antibody.

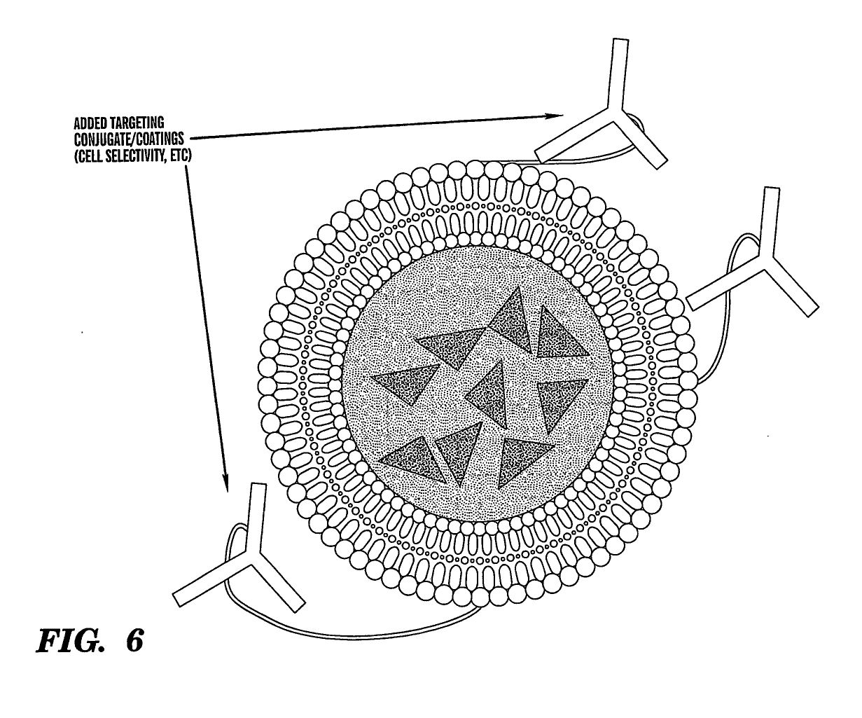

In another preferred embodiment, the nanoparticle may contain a targeting conjugate or coating, as depicted in FIG. 6.

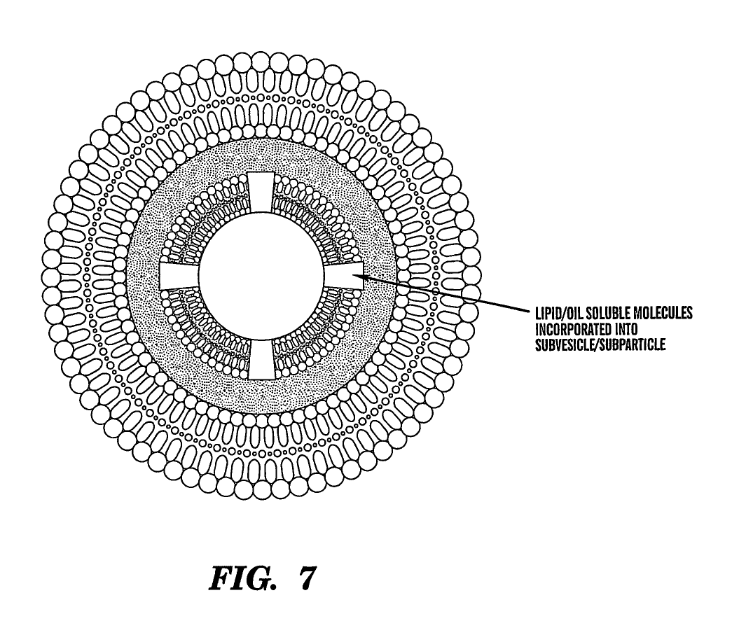

In another preferred embodiment, the nanoparticle may encapsulate a subvesicle or subparticle, as depicted in FIG. 7.

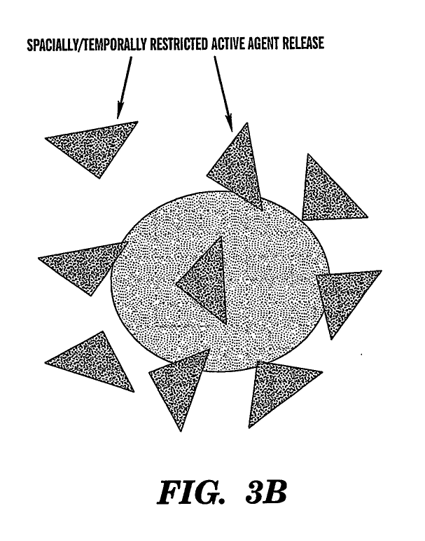

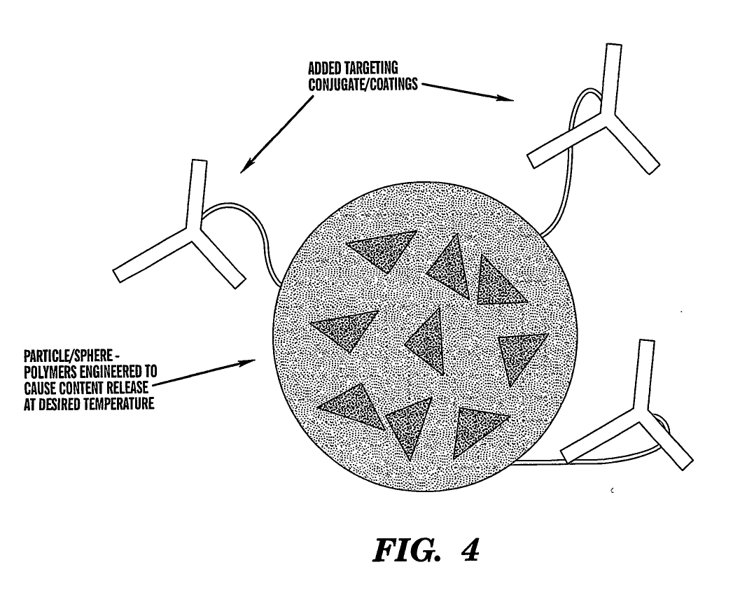

In another preferred embodiment, the particle may contain the desired agent embedded directly in a polymer engineered to cause content release at a desired temperature, as depicted in FIGS. 3-4. Preferably this configuration can also include added conjugate or coatings for targeting purposes.

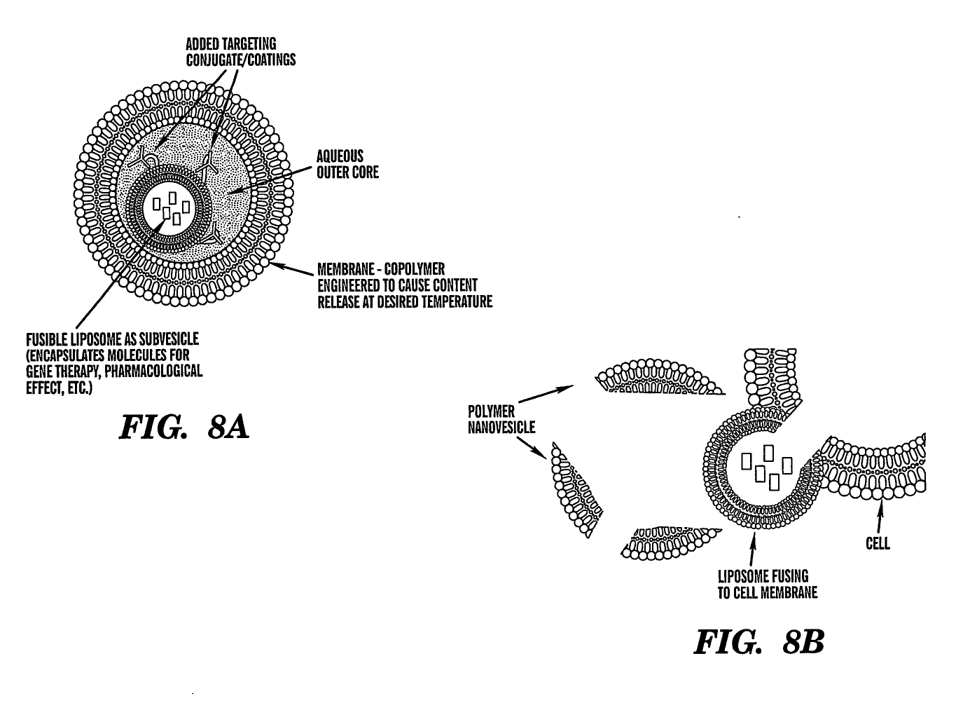

In another preferred embodiment, the nanoparticle may contain a fusible liposome subvesicle encapsulated within the aqueous core of a nanoparticle, as depicted in FIG. 8A. FIG. 8B depicts the delivery of the liposome's contents to a cell via membrane fusion.

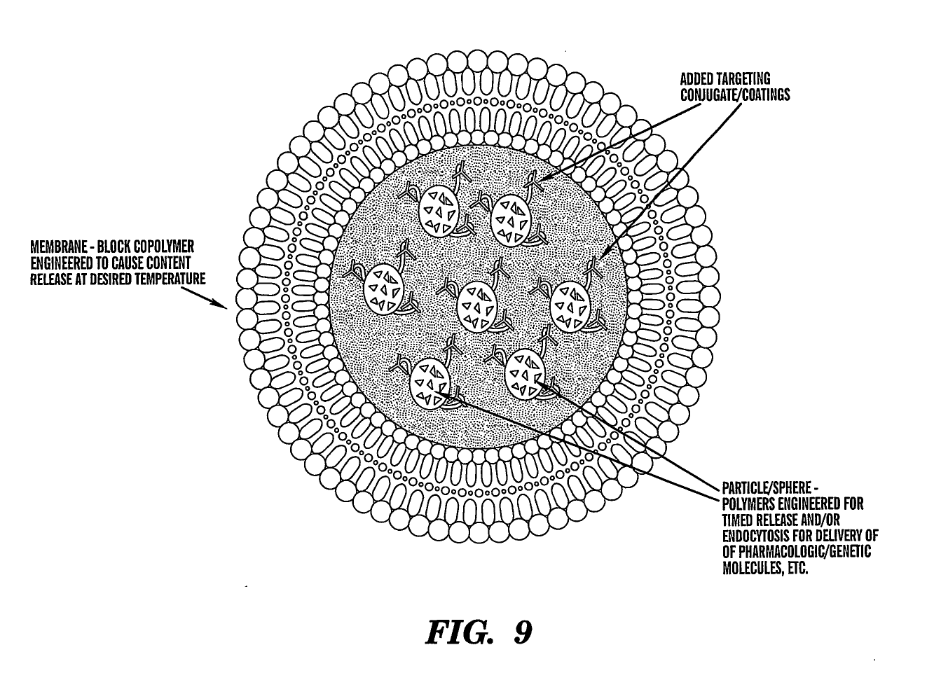

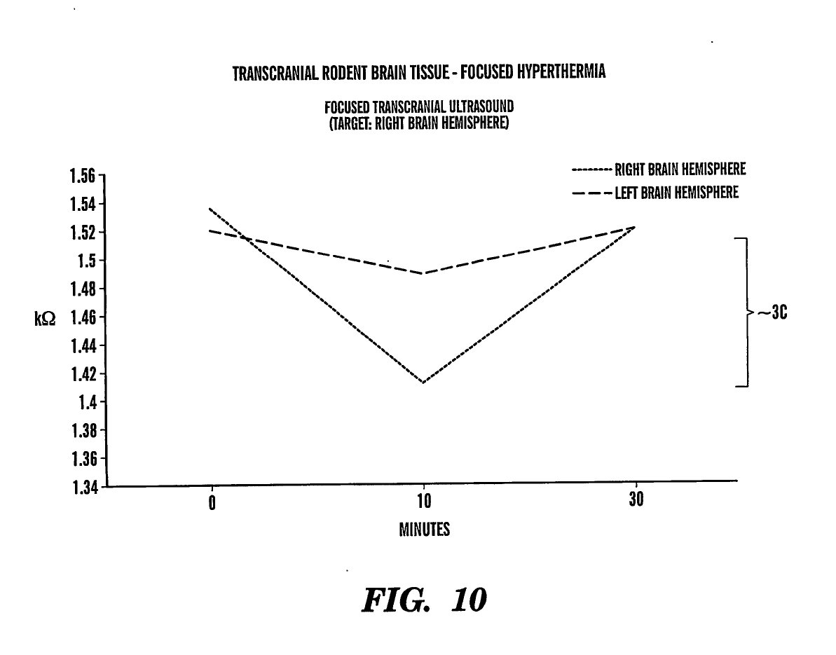

In another preferred embodiment, the particle may consist of a desired agent encapsulated in a polymer engineered for timed release and/or endocytic delivery within a nanovesicle engineered to cause content release at a desired temperature, as depicted in FIG. 9.

Substances which allow timed release of agents over extended periods of time are well known in the art. Preferably, the agent is released for days; even more preferably, for weeks.

In one particularly preferred embodiment, a biologically active agent is packaged with a timed release substance into a nanosphere or viral transfer vector; the nanosphere or viral transfer vector is then encapsulated into a thermosensitive nanovesicle, as depicted in FIG. 9. This embodiment allows localized release of the nanospheres and/or viral transfer vectors by focused ultrasound, followed by timed release of the agent of interest at the site of interest for extended periods of time. Preferably, the agent is released over a period of days, more preferably, the agent is released over weeks; even more preferably, the agent is released over months. Various molecules may also be attached to the surface of the nanospheres. For example, the nanospheres may include monoclonal antibodies to target specific tissues with the spatially and temporally restricted region(s).

In another preferred embodiment, the thermosensitive particle is a thermosensitive liposome, sometimes referred to as a liposome. Thermosensitive liposomes are known in the art. Liposomes according to the present invention may be prepared by any of a variety of techniques that are known in the art. See, e.g., U.S. Pat. No. 4,235,871; Published PCT applications WO 96/14057; New RRC, Liposomes: A practical approach, IRL Press, Oxford (1990), pages 33-104; Lasic D D, Liposomes from physics to applications, Elsevier Science Publishers, Amsterdam, 1993; Liposomes, Marcel Dekker, Inc., New York (1983). Entrapment of an active agent within liposomes of the present invention may also be carried out using any conventional method in the art. In preparing liposome compositions of the present invention, stabilizers such as antioxidants and other additives may be used as long as they do not interfere with the purpose of the invention. Examples include co-polymers of N-isopropylacrylamide (Bioconjug. Chem. 10:412-8 (1999)).

A method of preparing a liposomal formulation according to the present invention comprises mixing the bilayer components in the appropriate proportions in a suitable organic solvent, as is known in the art. The solvent is then evaporated to form a dried lipid film. The film is rehydrated (at temperatures above the phase transition temperature of the lipid mixture) using an aqueous solution containing an equilibrating amount of the surface active agent and a desired active agent. The liposomes formed after rehydration can be extruded to form liposomes of a desired size, as is known in the art. For example, where liposomes composed of 80:20 DPPC:MPPC are produced, rehydration is carried out at a temperature above the phase transition temperature of this particular lipid mixture (above 39.degree.C.). The aqueous solution used to rehydrate the lipid film comprises an equilibrating amount of lysolipid monomers (e.g., a concentration equal to the Critical Micelle Concentration of MPPC).

Polyethylene glycol (PEG) may be incorporated into the liposome bilayer to inhibit fusion with undesired membranes (Bulte et al., Proc. Intl. Soc. Mag. Reson. Med., Fifth Annual Meeting, p. 1596 (1997)).

The thermosensitive particle may include any other useful molecules. For example, the particle may include a monoclonal antibody on its surface which allows targeting of the particle to a desired site. For example, an antibody to the transferrin receptor, which can cross the blood-brain barrier, may be used to target particles to the brain.

In another preferred embodiment, any material which allows the particle to respond to a magnetic field may be incorporated into the particle. In this embodiment, the application of a magnetic field may then be used to localize the particles to a desired site (Bulte et al., Proc. Soc. Mag. Reson., Third Annual Meeting, p. 1139 (1995)). For example, membrane-colloidal magnetite (Fe304) may be incorporated into the liposome bilayer.

The thermosensitive particles may be administered to a subject using known means. Oral administration, injection, and inhalation are preferred routes for administration.

Focused Energy Sources

Any focused energy source, preferably a heat source capable of inducing highly localized hyperthermia to promote the destruction of the thermosensitive particles may be used. For example, focused ultrasound.

In another preferred embodiment the method of activation is governed by properties other than thermosensitivity, including but not limited to pH, gaseous cores and/or layers, metallic and/or magnetic particulate matter incorporated into said nanoparticle, nanoscale hydrophones sensitive to externally applied ultrasound frequency/wavelength and intensity, and external transcranial energy (e.g. ultrasound) controlled nanomechanical synthetic cells.

Active Agents

As used herein, an active agent “in the interior” or “entrapped within” or “encapsulated in” the particle is that which is contained in the particle. For example, an agent may be included within the interior space of a vesicle, compared to that partitioned into the polymer membrane or lipid bilayer and contained within the vesicle membrane itself. Similarly, an agent may be packaged into a nanosphere, which does not contain a interior space or cavity. As used herein, an active agent “within” or “entrapped within” or “encapsulated in” the membrane of a nanoparticle or a polymersome or lipid bilayer of a liposome is carried as a part of the membrane, as opposed to being contained in the interior space of the nanoparticle or liposome.

Active agents may be in any form suitable for use in nanoparticles or liposomes, as is known in the art. For example, aqueous solutions of active agents may be prepared for incorporation in particles. Aqueous solutions of active agents within the nanoparticles or liposomes of the present invention may be at the same osmotic pressure as that of the body fluid of the intended subject, or at an increased osmotic pressure (see U.S. Pat. No. 5,094,854); the aqueous solutions may also contain some precipitated active agent, as is known in the art. One preferred active agent for encapsulation in the interior of the nanoparticle or liposome is any water soluble, weak base agent.

The incorporation of certain active agents (such as some anesthetics) in nanoparticles or liposomes of the present invention may additionally alter (enhance or inhibit) the release of contents from the nanoparticle or liposome, or alter the transition temperature of the nanoparticle or liposome, compared to that which would be seen in a similar nanoparticle or liposome that did not contain the active agent.

Active agents suitable for use in the present invention include biologically active agents including therapeutic drugs, endogenous molecules, and pharmacologically active agents, including antibodies; nutritional molecules; cosmetic agents; diagnostic agents; and contrast agents for imaging. As used herein, an active agent includes pharmacologically acceptable salts of active agents. Suitable therapeutic agents include, for example, antineoplastics, antitumor agents, antibiotics, antifungals, anti-inflammatory agents, immunosuppressive agents, anti-infective agents, antivirals, anthelminthic, and antiparasitic compounds, including antibodies. Methods of preparing lipophilic drug derivatives which are suitable for nanoparticle or liposome formulation are known in the art (see e.g., U.S. Pat. No. 5,534,499 to Ansell, describing covalent attachment of therapeutic agents to a fatty acid chain of a phospholipid).

Preferred active agents suitable for use in the present invention include neuromodulatory agents. Preferred neuromodulators include NMDA or AMPA receptor agonists, GABA agonists, and sodium or calcium channel blockers. Certain preferred neuromodulators are listed in Table 1.

TABLE 1
PREFERRED NEUROMODULATORS

STIMULANTS (produce psychomotor arousal; treat attention deficit disorder)

| | | |
|---|---|---|
| Sympathomimetics | Dextroamphetamine | Monoamine (DA & NE) agonist; increase |
| | Methylphenidate | release; block re-uptake |
| Cholinomimetics | Nicotine | ACh agonist (high dose blocks) |
| | Muscarine | |
| Xanthines | Caffeine | Block adenosine receptors; GABA antagonist |
| | Theophylline | |
| Convulsants | Strychnine | Glycine antagonist |

DEPRESSANTS (produce sedation; treat pain, anxiety, sleep disorders)

| | | |
|---|---|---|
| Opioids | Morphine, Codeine | Endogenous opiate agonist |
| | Heroin, Methadone | |
| Barbiturates | Secobarbital | GABA agonist |
| Barbiturate-like | Meprobamate | Similar to barbiturates |
| Organic solvents | Alcohol | Disrupt neuronal membrane; may facilitate |
| | Ether | GABA |

HALLUCINOGENIC (produce distorted perception)

| | | |
|---|---|---|
| NE-like | Mescaline | Alter 5HT activity |
| 5HT-like | LSD | Alter 5HT activity |
| Other | Marijuana | Alter 5HT activity |
| | Anti-cholinergics, | |
| | PCP | |

Drugs Used to Treat Psychological Disorders

ANTIPSYCHOTICS (treat schizophrenia; also delirium and dementia)

| | | |
|---|---|---|
| Phenothiazines | Chlorpromazine | Block DA receptors |
| Butyrophenones | Haloperidol | Block DA receptors |
| Other | Clozapine | Block DA receptors |

ANTIDEPRESSANTS (treat depression and bipolar disorder)

| | | |
|---|---|---|
| Tricyclics (secondary amines) | Nortriptyline | Block NE re-uptake |
| Tricyclics (tertiary | Imipramine | Block 5HT re-uptake |
| amines) | Clomipramine | Block 5HT re-uptake |
| Heterocydics | Fluoxetine | Block NE & 5HT reuptake |
| MAO inhibitors | Pheneizine | Inhibit monoamine oxidase |
| | Tranylcypromine | |
| Lithium | Lithium | Stabilizes synapses |

ANTI-ANXIETY (treat acute and chronic anxiety; also sleep disturbances)

| | | |
|---|---|---|
| Benzodiazepines | Alprazolam | Faclitate GABA |
| | Diazepam | |
| Other | Buspirone | Decrease 5HT activity |

Notes:
ACh = acetyicholine
DA = dopamine
5HT = serotonin
GABA = gamma amino butyric acid
LSD = lysergic acid diethylamide
MAO = monoamine oxidase
NE = norepinephrine
PCP = phencyclidine

Other preferred active agents include gene expression modulating agents, including activators such as tetracycline for use with Tet-activated promoters (for example, in transgenic animals).

Still another preferred embodiment of the present invention provides for a method of delivery of nucleic acids, such as cDNAs, in targeted gene therapy treatments.

Another preferred class of active agents includes agents suitable for the treatment of stroke, including ischemic stroke. Examples of such preferred agents include thrombolytic agents such as tissue plasminogen activator or mannitol, or anticoagulants and antiplatelets such as warfarin, heparin, or aspirin. Such agents may be used in combination with other neuromodulators and/or neuroprotective agents.

In treating tumors or neoplastic growths, suitable compounds may include anthracycline antibiotics (such as doxorubicin, daunorubicin, carinomycin, N-acetyladriamycin, rubidazone, 5-imidodaunomycin, N30 acetyldaunomycin, and epirubicin) and plant alkaloids (such as vincristine, vinblastine, etoposide, ellipticine and camptothecin). Other suitable agents include paclitaxel (TAXOL.RTM.; a diterpenes isolated from the bark of the yew tree and representative of a new class of therapeutic agents having a taxane ring structure) and docetaxol (taxotere); mitotane, cisplatin, and phenesterine.

Anti-inflammatory therapeutic agents suitable for use in the present invention include'steroids and non-steroidal anti-inflammatory compounds, such as prednisone, methyl-prednisolone, paramethazone, 11-fludrocortisol, triamciniolone, betamethasone and dexamethasone, ibuprofen, piroxicam, beclomethasone; methotrexate, azaribine, etretinate, anthralin, psoralins; salicylates such as aspirin; and immunosuppresant agents such as cyclosporine. Antiinflammatory corticosteroids and the antiinflammatory and immunosuppressive agent cyclosporine are both highly lipophilic and are suited for use in the present invention.

Additional pharmacologic agents suitable for use in nanoparticles or liposomes of the present invention include anesthetics (such as methoxyflurane, isoflurane, enflurane, halothane, and benzocaine); antiulceratives(such as cimetidine); antiseizure medications such as barbituates; azothioprine (an immunosuppressant and antirheumatic agent); and muscle relaxants (such as dantrolene and diazepam).

Other preferred agents suitable for use in the present invention include molecules which promote bone healing. Such agents could be targeted for delivery to sites of bone fracture to reduce recovery time after an injury.

Imaging agents suitable for use in the present nanoparticle or liposome preparations include ultrasound contrast agents, radiocontrast agents (such as radioisotopes or compounds containing radioisotopes, including iodo-octanes, halocarbons, and renograf in), or magnetic contrast agents (such as paramagnetic compounds).

Nutritional agents suitable for incorporation into nanoparticles or liposomes of the present invention include flavoring compounds (e.g., citral, xylitol), amino acids, sugars, proteins, carbohydrates, vitamins and fat. Combinations of nutritional agents are also suitable.

Administration and Particle Size

Particles including polymer nanoparticles and liposomes of the present invention may be administered using methods that are known to those skilled in the art, including but not limited to oral administration, delivery into the bloodstream of a subject, inhalation, or subcutaneous administration of the particle. For example, the nanoparticles or liposomes may be administered by any suitable means that results in delivery of the nanoparticles or liposomes to the treatment site. It does not matter if the particle also goes to other sites because the agent will only be released where the energy source is directed. For example, nanoparticles or liposomes may be administered intravenously and thereby brought to the treatment site by the normal blood flow; it is the precise heating of the targeted site that results in the particle membranes being heated to the phase transition temperature so that the particle contents are preferentially released only at the site of the tumor.

Where treatment of a tumor or neoplasm is desired, effective delivery of a particle-encapsulated active agent via the bloodstream requires that the nanoparticle or liposome be able to penetrate the continuous (but “leaky”) endothelial layer and underlying basement membrane surrounding the vessels supplying blood to a tumor. Nanoparticles or liposomes of smaller sizes have been found to be more effective at extravasation into tumors through the endothelial cell barrier and underlying basement membrane which separates a capillary from tumor cells. See, e.g., U.S. Pat. No. 5,213,804 to Martin et al.

As used herein, “solid tumors” are those growing in an anatomical site other than the bloodstream (in contrast to blood-borne tumors such as leukemias) Solid tumors require the formation of small blood vessels and capillaries to nourish the growing tumor tissue.

It will further be appreciated that the particles of the present invention may be utilized to deliver of anti-infective agents to sites of infection, via the bloodstream. The use of for example, nanoparticles or liposomes containing a particle-forming lipid derivatized with a hydrophilic polymer, and having sizes ranging between 0.07 and 0.2 microns, to deliver therapeutic agents to sites of infection is described in published PCT patent application WO 93/19738. In accordance with the present invention, the anti-infective agent of choice is entrapped within a nanoparticle or liposome having a membrane according to the present invention, and the resulting particle formulation is administered parenterally to a subject, preferably by intravenous administration. If desired, localized hyperthermia may be induced at the site of infection to cause the preferential release of particle or liposomal contents at that site.

The size of particles in a preparation will depend upon the active agent contained therein and/or the intended target. Particles of between 0.05 to 0.3 microns in diameter are suitable for tumor administration (U.S. Pat. No. 5,527,528 to Allen et al.) Sizing of particles according to the present invention may be carried out according to methods known in the art, and taking into account the active agent contained therein and the effects desired (see, e.g., U.S. Pat. No. 5,225,212 to Martin et al; U.S. Pat. No. 5,527,528 to Allen et al). A preferred embodiment of the present invention is a particle of less than 10 microns in diameter, or a particle preparation containing a plurality e.g., liposomes of less than 10 microns in diameter. In a further preferred embodiment of the present invention, particles are from about 0.05 microns or about 0.1 microns in diameter, to about 0.3 microns or about 0.4 microns in diameter. Particle preparations may contain particles of different sizes.

In another preferred embodiment of the present invention, particles are from about 50 nm, 100 nm, 120 nm, 130 nm, 140 nm or 150 nm, up to about 175 mu, 180 nm, 200 nm, 250 nm, 300 nm, 350 nm, 400 nm or 500 nm in diameter.

In one aspect of the present invention, the particles are prepared to have substantially homogeneous sizes in a selected size range. One effective sizing method involves extruding an aqueous suspension of the particles through a series of polycarbonate membranes having a selected uniform pore size; the pore size of the membrane will correspond roughly with the largest sizes of liposomes produced by extrusion through that membrane. See e.g., U.S. Pat. No. 4,737,323 (Apr. 12, 1988).

In a further aspect of the present invention, particles are dispersed in physiological saline or PBS to provide an aqueous preparation of particles. For example the aqueous preparation may further include an equilibrating amount of the surface active agent contained in the liposome bilayer, to reduce or prevent loss of the surface active agent from the liposome bilayer into solution. Liposomes composed of DPPC:MPPC may be contained in physiological saline or PBS that contains from about 1 mmM to about 5 mM of MPPC monomer.

The amount of active agent to be entrapped within or carried by the thermosensitive particles according to the present invention will vary depending on the therapeutic dose and the unit dose of the active agent, as will be apparent to one skilled in the art. In general, however, the preparation of particles of the present invention is designed so that the largest amount of active agent possible is carried by the particle. Particles of the present invention may be of any type, however, LUVs are particularly preferred.

Subjects

The method of the present invention can be administered to any subject for which it would be desirable to locally deliver an active agent, including humans and animals. For example, the method of the present invention can be used to administer therapeutic agents to a patient in need thereof, as described further below. This invention also embraces treatment of animals, including for example animal models of disease.

Applications

The method of the present invention may be used for the localized delivery of a wide variety of agents to treat a wide variety of conditions.

One preferred embodiment of the present invention provides the delivery of neuromodulators to specific regions of the brain, to modulate neuronal transmission. Preferred embodiments of this invention include but are not limited to the delivery of inhibitory neurotransmitters to treat seizure foci in epileptics, including the application of thermosensitive nanoparticles that respond to natural thermal fluctuations present in surrounding neural tissue subject to epileptiform activity and without the use of any externally focused activation energy; excitatory neurotransmitters to treat Alzheimer's patients; neurotransmitters to enhance dopaminergic activity in Parkinson's patients; inhibitory neurotransmitters (such as BAPTA or MK-801) to prevent brain damage in stroke and post-traumatic neuronal cell loss victims, including emergency stroke treatment; and agents to treat developmental learning disabilities (such as ADHD).

Other preferred embodiments of the application of the invention within the central nervous system include but are not limited to localized delivery of agents to treat psychological disorders including for example schizophrenia and depression; enhancement of memory, learning and intelligence; treatment of cancer; post-traumatic neuronal cell loss including neurotoxicity and related cell damage common following severe brain insults; chronic and acute pain; eating disorders; sleep disorders; and targeted gene therapy.

The method of the present invention may also be used to deliver anti-arthritic agents such as anti-inflammatory drugs to sites of athritic lesions in arthritis patients.

Another embodiment prevents localized deposition of agents to treat artherosclerotic lesions.

In another preferred emboidment, the subject's brain is interfaced with a computer to modulate, extrapolate, or image neural information for any purpose (including but not limited to synthetic memory formation, and additional capabilities that require a system that can safely, directly, and noninvasively input and retrieve visual, auditory and integrated multimodal information directly into the human neural system—for example direct computer-brain interface based virtual reality).

In another embodiment, the present method may be used to deliver cytotoxic agents for localized tissue destruction, including for example solid tumors as well as undesired adipose tissue.

The method of the present invention may also be used for catalyzed tissue repair, including rapid bone fracture healing and localized deposition of coagulants to treat internal bleeding.

A further embodiment of the present invention provides the localized delivery of nucleic acids for targeted gene therapy.

### EXAMPLES

### Example 1

### Release of GABA from Lipid-Polymer Thermosensitive Nanovesicles

The neurotransmitter GABA has been incorporated into lipid polymer nanovesicles. The results of release of GABA from these lipid-polymer nanovesicles is depicted in Tables 2-5.

Spectrophotometer analysis using ninhydrin amino acid reagent was used to analyze the release of GABA (g-aminobutyric acid) which was encapsulated within lipid-polymer thermosensitive nanovesicles. These experiments demonstrate for the first time that neuromodulators can be encapsulated and their release controlled using the methods described herein. The following tables illustrate an exceptionally sharp GABA release curve and confirm the ability to exhibit precise control over concentrations and overall kinetics when releasing neuromodulators and other particles.

TABLE 2
DATA SET 02121

30 minute total reaction time
570 nm wavelength
120 nm diameter

| | Blank | Sup5 | RT | 38(3) | 38(15) | 41.5(3) | 41.5(15) |
|---|---|---|---|---|---|---|---|
| 1 | 0 | 0.0068 | 0.0053 | 0.0062 | 0.0099 | 0.2259 | 0.3207 |
| 2 | −0.0001 | 0.0048 | 0.0037 | 0.0042 | 0.0089 | 0.2269 | 0.3265 |
| 3 | −0.0002 | 0.0047 | 0.0037 | 0.0043 | 0.0092 | 0.2264 | 0.3266 |

| | 41.5(15) | (X − 100)*2 |
|---|---|---|
| 1 | 0.3207 | 0.8538 |
| 2 | 0.3265 | 0.855 |
| 3 | 0.3266 | 0.8482 |
| | Sup5 | Sup1 |
| 1 | 0.0068 | 3.1324 |
| 2 | 0.0048 | 3.3755 |
| 3 | 0.0047 | 3.4133 |

RT = Room Temp
Sup = Supernatant
## = Temp
(##) = Minutes of activation time

TABLE 3
DATA SET 02121

30 minute total reaction time
440 nm wavelength
120 nm diameter

| | Blank | Sup5 | RT | 38(3) | 38(15) | 41.5(3) | 41.5(15) |
|---|---|---|---|---|---|---|---|
| 1 | 0.001 | 0.0258 | 0.0228 | 0.025 | 0.0367 | 0.1395 | 0.1806 |
| 2 | 0.0003 | 0.0255 | 0.0224 | 0.025 | 0.0363 | 0.1397 | 0.1842 |
| 3 | 0.0003 | 0.0255 | 0.0225 | 0.0251 | 0.0362 | 0.139 | 0.1843 |

| | 41.5(15) | (X − 100)*2 |
|---|---|---|
| 1 | 0.1806 | 0.394 |
| 2 | 0.1842 | 0.3958 |
| 3 | 0.1843 | 0.394 |
| | Sup5 | Sup1 |
| 1 | 0.0258 | 2.435 |
| 2 | 0.0255 | 2.9121 |
| 3 | 0.0255 | 3.037 |

RT = Room Temp
Sup = Supernatant
## = Temp
(##) = Minutes of activation time

TABLE 4
DATA SET 02032

20 minute total reaction time
570 nm wavelength
120 nm diameter

| | Blank | RT | 37(3) | 37(15) | 42(3) | 42(15) |
|---|---|---|---|---|---|---|
| 1 | −0.006 | 0.0012 | 0.0014 | 0.0027 | 0.0619 | 0.0771 |
| 2 | −0.006 | 0.0012 | 0.0009 | 0.0012 | 0.0614 | 0.0778 |
| 3 | −0.008 | 0.0011 | 0.0012 | 0.0012 | 0.0614 | 0.0774 |
| | 42(15) | (X − 100)*2 | +TC,14 | | | |
| 1 | 0.0771 | 0.318 | | | | |
| 2 | 0.0778 | 0.312 | | | | |
| 3 | 0.0774 | 0.315 | | | | |
| | Blank | Sup 1 | Sup 2 | Sup 3 | Sup 4 | Sup 5 |
| 1 | −0.009 | 2.4414 | 2.2681 | 0.0022 | −0.0068 | −0.0066 |
| 2 | −0.0008 | 2.996 | 2.2971 | 0.0017 | −0.0072 | −0.0065 |
| 3 | −0.0008 | 3.2823 | 2.3123 | 0.002 | −0.0072 | −0.0069 |

RT = Room Temp
Sup = Supernatant
## = Temp
(##) = Minutes of activation time

TABLE 5
DATA SET 02032

20 minute total reaction time
440 nm wavelength
120 nm diameter

| | Blank | RT | 37(3) | 37(15) | 42(3) | 42(15) | (X-100)*2 |
|---|---|---|---|---|---|---|---|
| 1 | −0.001 | 0.0367 | 0.0406 | 0.0456 | 0.0845 | 0.0902 | 0.1724 |
| 2 | 0 | 0.0371 | 0.0405 | 0.0455 | 0.084 | 0.0894 | 0.1666 |
| 3 | −0.004 | 0.0372 | 0.0402 | 0.0459 | 0.0843 | 0.091 | 0.1708 |
| | Blank | Sup 1 | Sup 2 | Sup 3 | Sup 4 | Sup 5 | |
| 1 | −0.001 | 2.3135 | 1.0124 | 0.0426 | 0.0065 | 0.0012 | |
| 2 | 0 | 2.5399 | 1.0241 | 0.0428 | 0.0061 | 0.0012 | |
| 3 | −0.0004 | 2.8186 | 1.0305 | 0.0433 | 0.006 | 0.0015 | |

RT = Room Temp
Sup = Supernatant
## = Temp
(##) = Minutes of activation time

### Example 2

### Protocol Summaries for Animal Models

Protocol summary for the treatment of epilepsy in a rodent model. 1) Neural activity recorded while seizure induced in subject animal. 2) Subject injected with particle packaged inhibitory neurotransmitter. 3) Transcranial Focused Ultrasound tFUS) activated and focused on seizure foci. No external energy source is required for certain forms of epileptic activity. 4) Inhibitory neuromodulator released at seizure foci and epileptiform activity subdued.

Protocol summary for the treatment of Alzheimer's disease in a rodent model. 1) Subject animal bred with Alzheimer's dementia mutation. 2) Control, non-Alzheimer's, non-tFUS animal run through memory task. 3) Alzheimer's animal run through an identical memory task, demonstrating diminished task completion ability. 4) Subject injected with particle packaged neuromodulator (i.e. physotigimine, an AchE inhibitor). 5) tFUS targeted to hippocampal region of Alzheimer's subject. 6) Synthetic θ-rhythm induced in hippocampal formation of Alzheimer's subject, replacing function of deteriorated septal cholinergic cells, and enhancing memory retention. 7) Alzheimer's subject run through memory task under tFUS influence, demonstrating normal to exemplary task completion ability. Neural recording and histological analysis supplement all behavioral observations.

Protocol summary for treatment of Parkinson's disease in a rodent. 1) Animal with Parkinson's disease used as experimental subject. 2) Control, non-Parkinson's, non-tFUS animal run through a motor function related task. 3) Parkinson's animal run through an identical memory task, demonstrating diminished task completion ability. 4) Subject injected with particle packaged excitatory neuromodulator (e.g. dopamine). 5) tFUS targeted to Parkinson's affected brain region—e.g. substantia nigra (pars compacta)—of Parkinson's subject, enhancing dopaminergic activity, and demonstrating normal task completion ability. 6) tFUS also focused on pathways utilized by aforementioned region to modulate other areas of the basal ganglia and premotor cortex, further alleviating the tremor and inability to initiate movement prevalent in Parkinson's patients. Neural recording and histological analysis supplement all behavioral observations.

Protocol summary for treatment of ischemic Stroke in a rodent model. 1) Blood flow to small population of subject animal neurons blocked, resulting in immediate necrosis in localized region. 2) Target release of neuroprotective, thrombolytic, and other such agents which suppress rampant activity and encourage blood flow to surrounding damaged areas prevents disabling brain damage by reinstating oxygen supply and controlling neural firing in areas affected by the stroke infarction. Depositing local modulators (e.g. BAPTA or MK-801 to disrupted glutamate cascade and t-PA to reinstate blood flow) would prevent brain damage to critical brain regions while allowing the patient to sustain activity in neural systems controlling heart function, breathing, and the like during the healing process. Preemptive measures can also be employed by depositing anti-coagulants (such as warfarin), anti-platelets (such as aspirin), and other such agents in order to thin blood only in body regions at risk of future infarction. Neural recording and histological analysis supplement all behavioral observations.

Protocol summary for hemorrhagic stroke treatment. 1) Hemorrhagic stroke induced in subject animal. 2) Coagulants and other agents, including but not limited to medication that helps to protect brain cells such as Hydergine, an antioxidant, Piracetam, a nootropic medication similar to pyroglutamate, antioxidant nutrients, drugs, and hormones, along with specific calcium channel blockers and cell membrane stabilizing agents—which encourage blood clotting and/or protect cells, targeted to region of concern, halting potentially lethal internal bleeding. Neural recording and histological analysis supplement all behavioral observations.

Protocol summary for treatment of DLD in a rodent model. 1) Monitor subject animal with DLD (e.g. ADD/ADHD) for hyperactivity during behavioral task. 2) Control subject is injected with ADD/ADHD medication and monitored for side effects. 3) Test subject injected with particle packaged ADD/ADHD medication. 4) tFUS targeted to area relevant to ADD/ADHD cause. 5) Medication released only in necessary areas, reducing and eliminating adverse side effects common to ADD/ADHD medication use. 6) Subject run through behavioral task during and under and after treatment, demonstrating hyperactivity extinction. Neural recording and histological analysis supplement all behavioral observations.

Protocol summary for treatment of Post-Traumatic Neuronal Cell Loss in a rodent model. 1) Subject animal with Neurotoxicity monitored during behavioral task. 2) Subject injected with particle packaged neurotransmitter. 3) tFUS targeted to damaged areas. 4) Excitability and activity manipulated to reestablish normal plasticity in affected brain regions. 5) Subject run through behavioral task, demonstrating reestablished brain function. Neural recording and histological analysis supplement all behavioral observations.

Protocol summary for improving hLTP in a primate model. 1) Subject run through memory task; completion capability recorded. 2) Subject injected with particle packaged excitatory neuromodulator (e.g. delivery of physostigmine to the medial septum to induce theta in the hippocampus and enhance encoding during a task learning period). 3) tFUS targeted to hippocampus. 4) θ-rhythm oscillation modulated, reinforcing septal inputs and allowing control over stimuli retention. 5) Subject run through memory task under tFUS influence, demonstrating increased stimuli retention, associational ability, and learning speed. Neural recording and histological analysis supplement all behavioral observations.

Protocol summary for removal of learning ability in a primate model. 1) Subject run through memory task; completion capability recorded. 2) Subject injected with particle packaged inhibitory neurotransmitter. 3) tFUS targeted to hippocampus. 4) Subject run through different memory task (of same difficulty) under LTD inducing tFUS influence, demonstrating diminished stimuli retention, and temporary loss of learning ability. Neural recording and histological analysis supplement all behavioral observations.

Protocol summary for memory erasure in a primate model. 1) Subject run through multi-trial memory task; increasing completion speed recorded. 2) Subject injected with particle packaged inhibitory neurotransmitter. 3) tFUS targeted to hippocampus; regional LTD induced. 4) Subject run through same memory task; completion capability recorded, demonstrating loss of previously gained memory. Though initial experimentation is confined to the hippocampus, plasticity modulation is not limited to any single brain region. In fact, expanding hLTP/hLTD manipulation to other regions will enhance desired effects, and is a natural next step. Neural recording and histological analysis supplement all behavioral observations.

Protocol summary for treatment of Arthritis in an animal model. 1) Subject animal with arthritis injected with particle packaged, arthritis-killing arthritic medication. 2) tFUS targeted to arthritis location. 3) High potency drug selectively released, demonstrating arthritis treatment with insignificant or zero damage to surrounding cells. Though arthritis is destroyed in the this experiment, this procedure is not limited to a particular condition. Any spatially localized disease can be annihilated by this drug delivery method (e.g. cancer). Neural recording and histological analysis supplement all behavioral observations.

Protocol summary for targeted adipose tissue destruction in an animal model. 1) Overweight subject is injected with particle packaged, fat-cell-killing compound. 2) tFUS is targeted to unwanted fat excess, demonstrating localized fat annihilation. Neural recording and histological analysis supplement all behavioral observations.

All references described herein are incorporated herein by reference.

What is claimed is:

1. A method for non-invasive localized delivery of biologically active molecules to a region of a subject, comprising packaging a molecule(s) of interest inside an ultrasound-responsive particle, administering said particles to a subject, and inducing localized release of said molecules from said particles in the region using a focused energy source.

2. The method of claim 1, wherein the ultrasound-responsive particle of the invention is selected from the group consisting of thermosensitive nanoparticles, thermosensitive liposomes, thermosensitive nanovesicles, thermosensitive polymersomes and thermosensitive nanospheres.

3. The method of claim 1, wherein the molecules are released from the particles using any non-invasive method which induces localized hyperthermia, including focused ultrasound.

4. The method of claim 1, wherein the subject is a human.

5. The method of claim 1, wherein the subject is an animal.

6. The method of claim 1, wherein a nanosphere or a viral vector is encapsulated within the particle.

7. The method of claim 6, wherein the nanosphere or viral vector contains an agent for prolonged release over an extended period of time or interval release.

8. The method of claim 1, wherein the particles are administered to the subject by a route selected from the group consisting of oral administration, intravenous administration, and administration via a nebulizer.

9. The method of claim 1, wherein the subject is in need of treatment for a neural condition.

10. The method of claim 9, wherein the particles are coated with any composition which promotes or enhances transport of the particles across the blood brain barrier.

11. The method of claim 10, wherein the composition which promotes or enhances transport of the particles across the blood brain barrier is selected from the group consisting of Polysorbate 80/85 and antibodies which allow transport across the blood brain barrier.

12. The method of claim 11, wherein the antibody is an anti-transferrin receptor antibody.

13. The method of claim 9, wherein the method is selected from the group consisting of epilepsy, Alzheimer's disease, Parkinson's disease, stroke, pain management, depression, mental illness, psychological disorders, developmental learning disabilities, and post-traumatic neuronal cell loss.

14. The method of claim 1, wherein the subject is in need of treatment for a condition selected from the group consisting of arthritis, cancer, heart disease, bone fracture, and internal bleeding.

15. The method of claim 1, wherein the subject is in need of treatment for a condition which can be treated by targeted gene therapy, and the molecule of interest is a nucleic acid.

16. The method of claim 1, wherein the subject's brain functionality is influenced or enhanced, including learning ability, memory alteration, perception of pain, sleep/waking state, emotion, hunger and libido.
